(19) United States
(12) Patent Application Publication
(10) Pub. No.: US 2024/0001091 A1
(43) Pub. Date: Jan. 4, 2024

(54) HIGH PRESSURE INFLATION DEVICE

(71) Applicant: Merit Medical Systems, Inc., South Jordan, UT (US)

(72) Inventors: Michael Dean Haslam, Sandy, UT (US); Richard P. Jenkins, Bluffdale, UT (US); David Cise, Herriman, UT (US)

(21) Appl. No.: 18/344,562

(22) Filed: Jun. 29, 2023

Related U.S. Application Data
(60) Provisional application No. 63/367,572, filed on Jul. 1, 2022.

(51) Int. Cl. A61M 25/10

(52) U.S. Cl. CPC A61M 25/10182

(57) ABSTRACT

Inflation devices and methods used to inflate a balloon of a balloon catheter are disclosed. The inflation devices include a pressure member to contain a fluid to be pressurized. The pressure member includes a load transfer orifice configured to receive a load transfer member of a threaded insert. The load transfer member can transfer an axial load applied to the threaded insert during pressurization of the fluid to the pressure member. A plunger including a thread rail is slidingly disposed within the pressure member. The thread rail is engaged with the threaded insert when the fluid is pressurized. An actuator disengages the thread rail from the threaded insert to depressurize the fluid.

### RELATED APPLICATIONS

This application claims priority to U.S. Provisional Application No. 63/367,572, filed on Jul. 1, 2022 and titled “High-Pressure Inflation Device” which is hereby incorporated by reference in its entirety.

### TECHNICAL FIELD

The present disclosure relates generally to devices used to pressurize, depressurize, or otherwise displace fluid, particularly in medical devices. More specifically, the present disclosure relates to high-pressure devices used to pressurize, depressurize, or otherwise displace fluid along a fluid line in order to inflate or deflate a medical device, such as a balloon.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of an inflation device.

FIG. 2 is an exploded view of the inflation device of FIG. 1.

FIG. 3 is a side cross-sectional view of the inflation device of FIG. 1.

FIG. 4 is a perspective view of an embodiment of a pressure member of the inflation device of FIG. 1.

FIG. 5A is a perspective view of an embodiment of a plunger tip of the inflation device of FIG. 1.

FIG. 5B is a side cross-sectional view of the plunger tip of FIG. 5A.

FIG. 6 is a perspective view of an embodiment of a threaded insert of the inflation device of FIG. 1.

FIG. 7 is a perspective view of the threaded insert of FIG. 6 coupled to the pressure member of FIG. 4.

FIG. 8 is a perspective view of an embodiment of a grip of the inflation device of FIG. 1

FIG. 9A is a bottom perspective view of an embodiment of a plunger of the inflation device of FIG. 1.

FIG. 9B is a top perspective view of the plunger of FIG. 9A.

FIG. 9C is a cross-sectional view of the plunger of FIG. 9B through section line 9C-9C.

FIG. 10A is a side cross-sectional view of the plunger of FIG. 9A and the threaded insert of FIG. 7 in an engaged position.

FIG. 10B is a side cross-sectional view of the plunger of FIG. 9A and the threaded insert of FIG. 7 in a disengaged position.

FIG. 11A is a perspective view of an embodiment of an actuator of the inflation device of FIG. 1.

FIG. 11B is a top view of the actuator of FIG. 11A.

FIG. 12A is a partial cross-sectional view of the inflation device of FIG. 1 in a pressurization state.

FIG. 12B is a partial cross-sectional view of the inflation device of FIG. 1 in a depressurization state.

### DETAILED DESCRIPTION

In certain instances, an inflation device is in fluid communication with a balloon disposed at an end of a catheter. The inflation device may be used to generate a high pressure to inflate the balloon for a variety of medical procedures. For example, the inflation device can be used to widen a stricture of a vessel or passage, expand a stent within a vessel or passage, or occlude a vessel or passage.

The inflation device may include a syringe that utilizes threads to advance or retract a plunger by rotating the plunger relative to the body of the syringe such that the threads cause longitudinal displacement of the plunger relative to the body. In some instances, an inflation device may further include retractable threads, enabling a practitioner to disengage the threads and displace the plunger by simply pushing or pulling the plunger. The inflation device may comprise a threaded insert configured to constrain movement of the plunger within the syringe body. The threaded insert may comprise threads configured to engage with the retractable threads.

An embodiment of an inflation device within the scope of this disclosure includes a pressure member having a load transfer orifice. A threaded insert is coupled to the pressure member. The threaded insert includes a load transfer member that is disposed within the load transfer orifice and internal threads. A plunger is slidingly disposed within the pressure member and includes a thread rail selectively coupled to the threaded insert. The thread rail includes threads to engage with the threads of the threaded insert and protrusions. A plunger tip is operably coupled to a distal end of the plunger. An actuator includes a guide member coupled to the thread rail. The guide member includes ramps and slots configured to engage with the protrusions of the thread rail.

When the inflation device disclosed within this disclosure is pressurized to inflate a balloon, the protrusions of the thread rail engage the ramps of the guide member causing the threads of the thread rail to engage with the threads of the threaded insert. A handle of the actuator is rotated by a user causing the plunger tip to be displaced distally and pressurize fluid within the pressure member. An axial load applied to the threads is transferred to the pressure member through the load transfer member of the threaded insert and the load transfer orifice of the pressure member. When the inflation device is depressurized to deflate the balloon, the handle of the actuator is moved distally relative to the plunger causing the protrusions to be disposed in the slots of the guide member. This allows the thread rail to be radially inward displaced and the threads of the thread rail to disengage from the threads of the threaded insert. The disclosed embodiment of the inflation device allows the inflation device to be pressurized to a high pressure while reducing a force needed for activation of depressurization.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

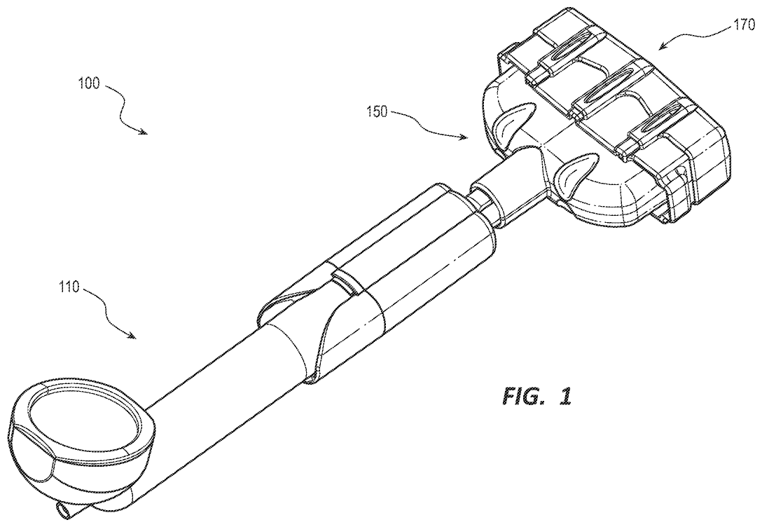

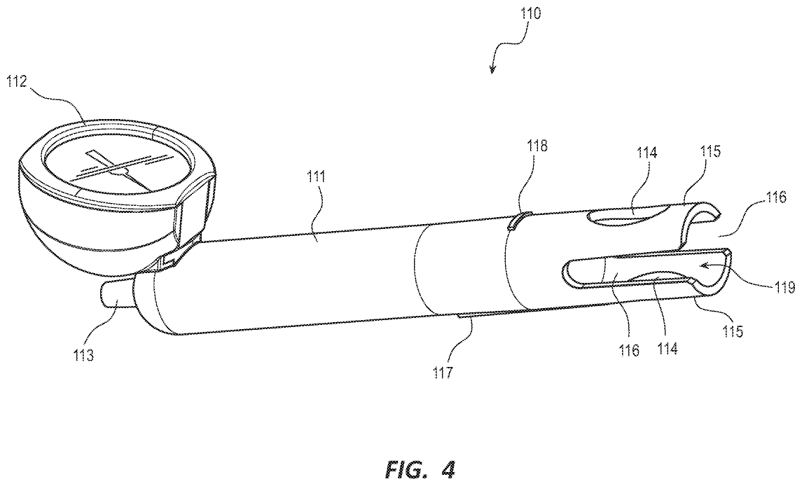

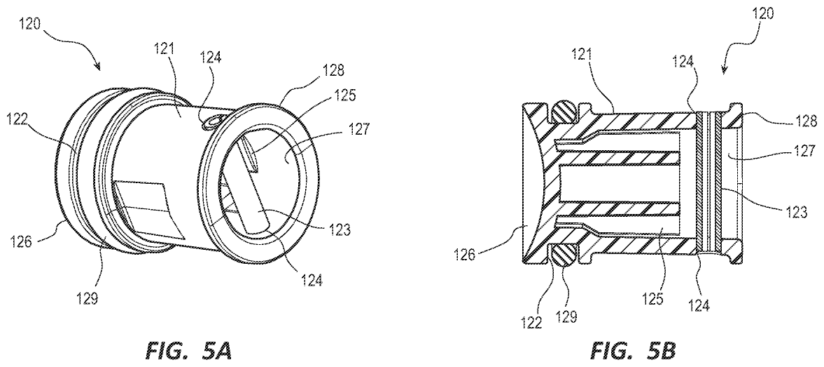

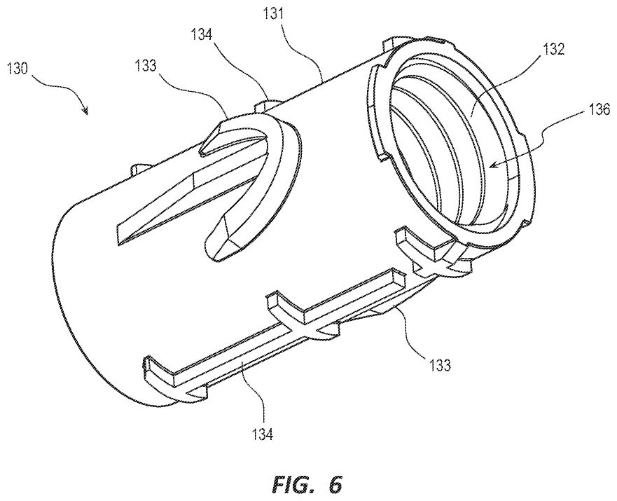

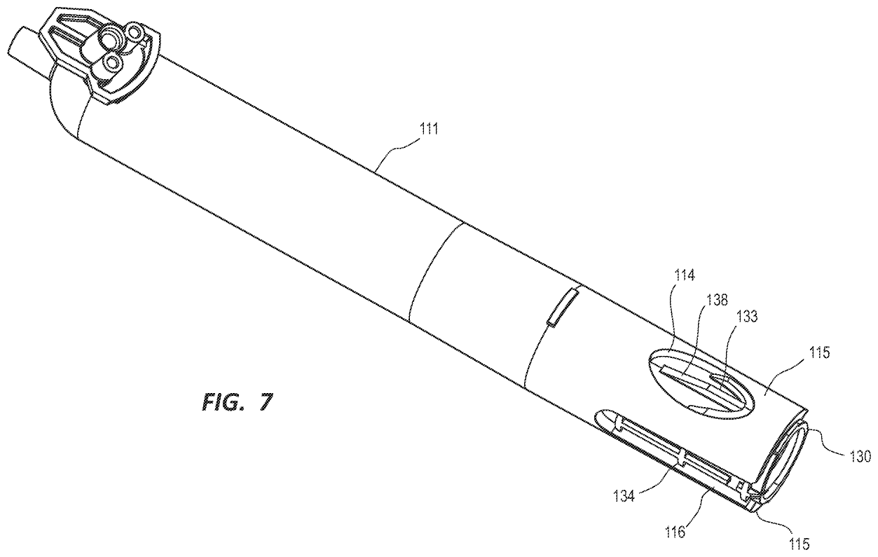

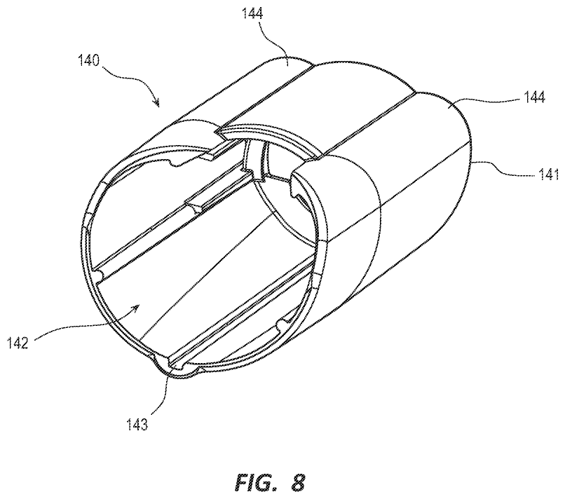

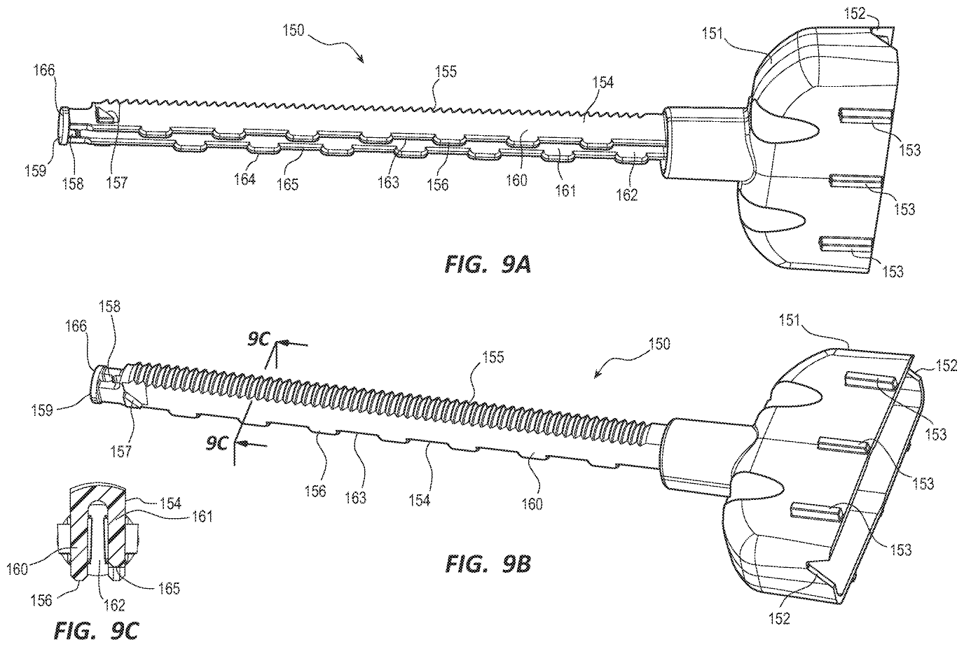

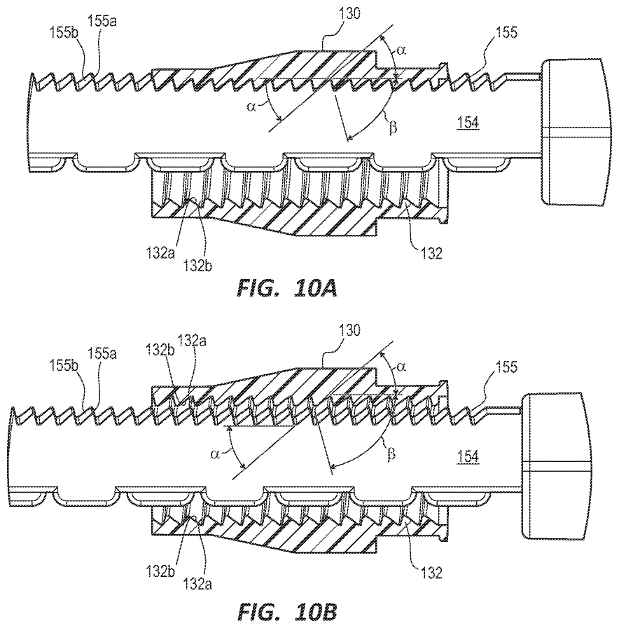

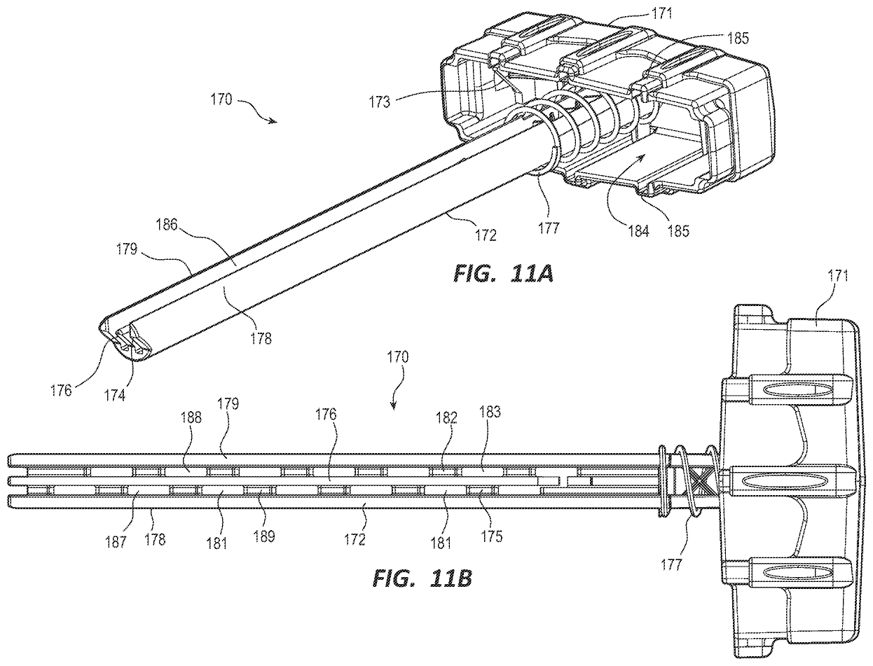

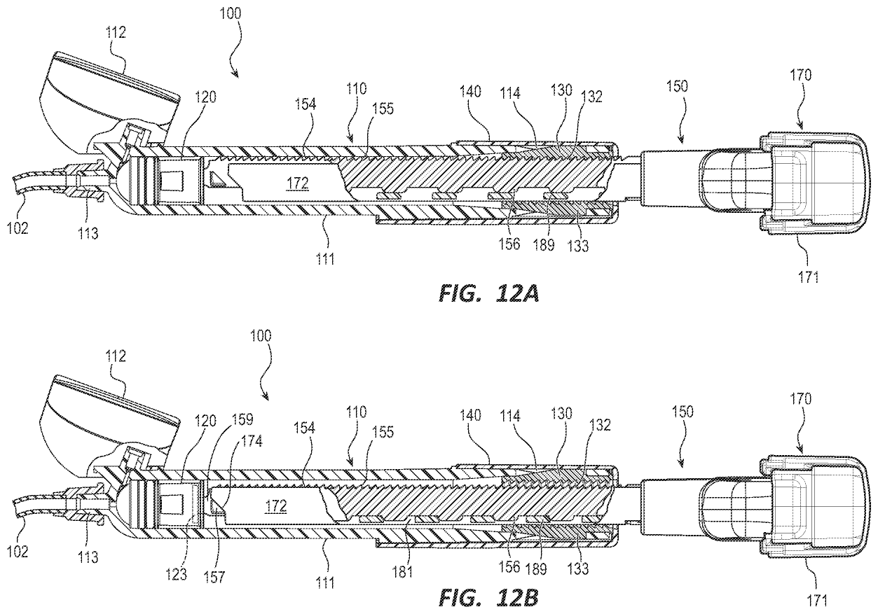

FIGS. 1-3 illustrate different views of an embodiment of an inflation device. FIG. 4 illustrates an embodiment of a fluid reservoir of the inflation device. FIGS. 5A and illustrate an embodiment of a plunger tip of the inflation device. FIG. 6 illustrates an embodiment of a threaded insert of the inflation device. FIG. 7 illustrates the threaded insert coupled to the fluid reservoir. FIG. 8 illustrates an embodiment of a grip of the inflation device. FIGS. 9A-9C illustrate an embodiment of a plunger of the inflation device. FIG. 10A illustrates the plunger and the threaded insert in an engaged state. FIG. 10B illustrates the plunger and the threaded insert in a disengaged state. FIGS. 11A and 11B illustrate an embodiment of an actuator of the inflation device. FIG. 12A illustrates the inflation device in a pressurization state. FIG. 12B illustrates the inflation device in a depressurization state. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

FIG. 1 illustrates an embodiment of an inflation device 100. As illustrated in FIG. 1, the inflation device 100 can include three broad groups of components; each group may have numerous subcomponents and parts. The three broad component groups are: a pressure member 110, a plunger 150, and an actuator 170.

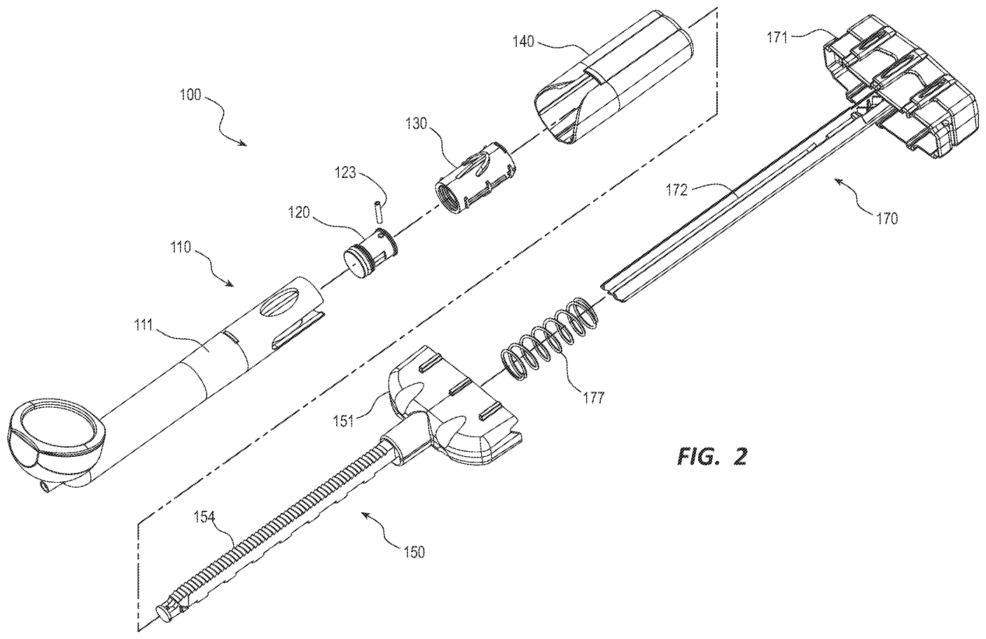

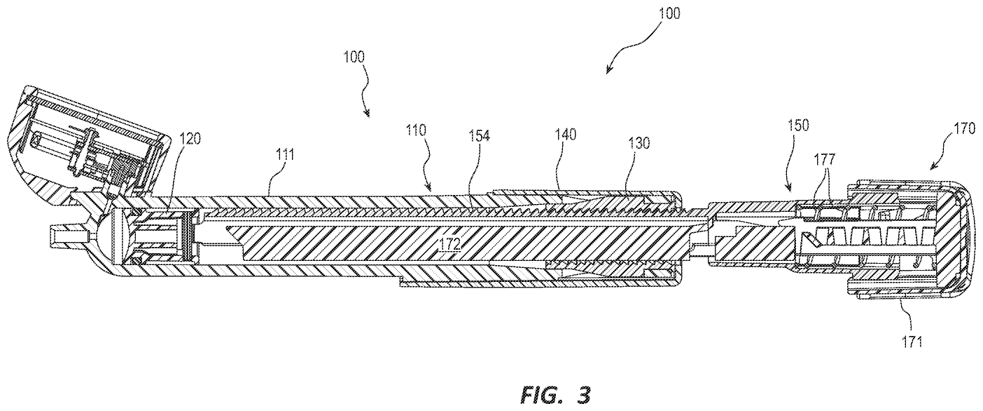

FIGS. 2 and 3 illustrate the embodiment of the inflation device 100. As illustrated, the pressure member 110 may include a fluid reservoir 111, a plunger tip 120 slidingly disposed within the fluid reservoir 111, a threaded insert 130 coupled to the fluid reservoir 111, and a grip 140 coupled to an exterior of the fluid reservoir 111. Further, as illustrated in FIG. 2, the plunger 150 may include a trigger grip 151 and a thread rail 154 extending distally from the trigger grip 151. Further still, as illustrated in FIG. 2, the actuator 170 can include a handle 171, a plunger guide member 172 extending distally from the handle 171, and a compliant member 177 disposed around the plunger guide member 172.

FIG. 4 illustrates an embodiment of the fluid reservoir 111 of the pressure member 110. As illustrated in the embodiment, the fluid reservoir 111 can include a cylindrical shape that defines a pressure chamber 119. The pressure chamber 119 may have a diameter ranging from about 15.2 millimeters to about 30.5 millimeters and may be about 16.3 millimeters. The fluid reservoir 111 may be formed of a rigid material capable of withstanding a high pressure without fracturing or deforming. For example, the fluid reservoir 111 can be formed of polycarbonate, glass, metal, copolyester, nylon, cyclic olefin polymer, or cyclic olefin copolymer. Other materials are contemplated. A nozzle 113 may be disposed at a distal end and may be in fluid communication with the pressure chamber 119. In some embodiments, the nozzle 113 is a male Luer fitting. The nozzle 113 can be configured to couple an inflatable medical device to the fluid reservoir 111. Fluid from the pressure chamber 119 can flow through the nozzle 113 and into the inflatable medical device causing the inflatable medical device to inflate. Fluid can also flow from the inflatable medical device through the nozzle 113 and into the pressure chamber 119 allowing the inflatable medical device to deflate. In certain embodiments, the inflatable medical device is a balloon.

A pair of longitudinal flex members 115 may be disposed at a proximal end of the fluid reservoir 111. Slots 116 can be disposed between the flex members 115 to allow the flex members 115 to deflect radially inward and outward. A load transfer orifice 114 can be disposed in each of the flex members 115. The load transfer orifice 114 may be sized and shaped to receive a load transfer member of the threaded insert 130, as will be described below. The flex members 115 can flex radially outward over the threaded insert 130 when the threaded insert 130 is coupled to the fluid reservoir 111. One or more longitudinal ribs 117 may be disposed adjacent the flex members 115. The rib 117 can prevent rotation of the grip 140 relative to the fluid reservoir 111.

Optionally or alternatively, a pressure gage 112 can be coupled to the fluid reservoir 111 such that the pressure gage 112 is in fluid communication with the pressure chamber 119. The pressure gage 112 may be of any suitable type to measure a fluid pressure within the fluid reservoir 111. For example, the pressure gage 112 can be an analog pressure gage or a digital pressure gage. Other types of pressure gages are contemplated.

FIGS. 5A and 5B illustrate an embodiment of the plunger tip 120. As illustrated in FIGS. 5A and 5B, the plunger tip 120 can include a body 121 having a cylindrical shape. The plunger tip 120 may be formed of any suitable material, such as polycarbonate, high density polyethylene, polypropylene, acrylonitrile butadiene styrene, nylon, polyoxymethylene, polysulfone, polyetheretherketone, etc. In some embodiments, the materials may be reinforced or filled with a filler, such as glass. A circumferential channel 122 can be disposed adjacent a distal end 126. An O-ring 129 may be disposed within the channel 122 to create a seal between the body 121 and an interior surface of the fluid reservoir 111. The distal end 126 may be concave to reduce air bubbles within the fluid reservoir 111 to allow for easier removal of air bubbles from the fluid reservoir during priming of the inflation device 100. For example, a transition angle from the distal end 126 to the interior surface of the fluid reservoir 111 can be greater than 90 degrees to substantially prevent air bubbles from being trapped between the distal end 126 and the interior surface of the fluid reservoir 111. A bore 127 can be open at a proximal end 128 and closed at the distal end 126. An engagement member 125 may be disposed within the bore 127 to engage a distal end of the thread rail 154. The engagement member 125 can include a proximal face oriented perpendicular to a longitudinal axis of the body 121. A coupling pin 123 may be disposed through a pin hole 124 disposed adjacent the proximal end 128 to operatively couple the thread rail 154 to the plunger tip 120. The coupling pin 123 can be a spring pin or any other suitable type of pin.

FIG. 6 illustrates an embodiment of the threaded insert 130. As illustrated, the threaded insert 130 can include a cylindrical body 131 having a bore 136 therethrough. The threaded insert 130 may be formed of rigid or semi-rigid polymeric material. For example, the material can be polycarbonate, high density polyethylene, polypropylene, nylon, acrylonitrile butadiene styrene, nylon, polysulfone, polyetheretherketone or polyoxymethylene. In some embodiments, the materials may be reinforced or filled with a filler, such as glass. Other materials are contemplated. The bore 136 may include female helical insert threads 132 extending along a length of the bore 136. The insert threads 132 may include either a single start or a double start. A load transfer member 133 may be disposed on opposing sides of an exterior surface of the body 131. The load transfer member 133 can have an arcuate shape to transfer an axial load from the threaded insert 130 to the pressure member 110. In other embodiments, the load transfer member 133 can include any other suitable shape. Further, a slot feature 134 can be disposed on the exterior surface of the body 131 between the load transfer members 133.

FIG. 7 illustrates the threaded insert 130 coupled to the fluid reservoir 111. As illustrated, the threaded insert 130 can be disposed within the fluid reservoir 111 with the load transfer member 133 disposed within the load transfer orifice 114. The slot feature 134 may be disposed within the slot 116 to guide the threaded insert 130 into the fluid reservoir 111. The load transfer member 133 may include a ramp 138 to facilitate disposing of the load transfer member 133 within the load transfer orifice 114. The ramp 138 can deflect the flex member 115 radially outward as the threaded insert 130 is inserted into the fluid reservoir 111.

FIG. 8 illustrates an embodiment of the grip 140. As illustrated, the grip 140 can include a body 141 having a bore 142 extending therethrough. The body 141 may be formed of a rigid or semi-rigid material, such as polycarbonate, polyethylene, polypropylene, polyurethane, acrylonitrile butadiene styrene, nylon, polyoxymethylene, or thermoplastic elastomer. Other materials are contemplated. A longitudinal channel 143 can be disposed within the bore 142 to receive the rib 117 of the fluid reservoir 111. When the rib 117 is disposed within the channel 143, the grip 140 is prevented from rotating relative to the fluid reservoir 111. The grip 140 may have an ergonomic shape or surface to allow a user to grasp the grip 140 in a hand to prevent the fluid reservoir 111 from rotating when the actuator 170 is rotated. For example, in the illustrated embodiment, the grip 140 includes lobes 144 to provide an ergonomic grasping shape. In other embodiments, an exterior surface of the grip 140 can include bumps, divots, ribs, knurls, texturing, or a compliant material. Other ergonomic shapes and surfaces are contemplated within the scope of this disclosure.

FIGS. 9A-9C illustrate an embodiment of the plunger 150. As illustrated, the plunger 150 can include a trigger grip 151 and a thread rail 154. The plunger 150 may be formed of a rigid or semi-rigid material, such as polycarbonate, high density polyethylene, polypropylene, nylon, acrylonitrile butadiene styrene, nylon, polysulfone, polyetheretherketone or polyoxymethylene. In some embodiments, the materials may be reinforced or filled with a filler, such as glass. Other materials are contemplated. The trigger grip 151 may include a graspable shape, including finger grips, to be grasped by fingers of the user. A proximal plunger ramp 152 can be disposed adjacent a proximal end of the trigger grip 151 to interface with a proximal actuator ramp of the actuator 170, as will be discussed below. Longitudinal ribs 153 can be disposed on an exterior surface of the trigger grip 151 to maintain axial alignment of the trigger grip 151 with the handle 171 when the handle 171 is displaced over the trigger grip 151.

The thread rail 154 can extend distally from the trigger grip 151. As illustrated, the thread rail 154 may include a first rail 160 and a second rail 161 that extend parallel to each other. A channel 162 is disposed between the rails 160, 161. Each of the rails 160, 161 can include protrusions 156, 164 respectively, extending downward from the rails 160, 161. The protrusions 156 of the first rail 160 may be separated by gaps 163 and the protrusions 164 of the second rail 161 may be longitudinally separated by gaps 165. Further, the protrusions 156 of the first rail 160 can be longitudinally offset relative to the protrusions 164 of the second rail 161. In other words, as shown in FIG. 9C, a transverse cross-section of the thread rail 154 through section 9B-9B shows the protrusion 156 of the first rail 160 transversely aligned with a gap 165 of the second rail 161.

A distal plunger ramp 157 may be disposed adjacent a distal end 159 of the thread rail 154 to be engaged by a distal actuator ramp of the actuator 170, as will be discussed below. A pin passage 158 can be disposed through the thread rail 154 adjacent the distal end 159 to receive the coupling pin 123 of the plunger tip 120. The distal end 159 can include a flat head 166 to press against the engagement member 125 of the plunger tip 120 when the plunger 150 is displaced distally to pressurize the fluid reservoir 111. Further, the flat head 166 can slidingly interface with the engagement member 125 when the thread rail 154 is disengaged from the threaded insert 130.

The thread rail 154 can include male plunger threads 155 disposed along a length of the thread rail 154 to selectively engage with the insert threads 132 of the threaded insert 130. The plunger threads 155 may include an arc length of from about 45 degrees to about 60 degrees and may be about 52 degrees.

FIGS. 10A and 10B illustrate two operable positions of the thread rail 154 with respect to the threaded insert 130. FIG. 10A shows the thread rail 154 disposed in an engaged position, such that the plunger threads 155 are engaged with the insert threads 132. FIG. 10B shows the thread rail 154 in a disengaged position, wherein the thread rail 154 is sufficiently retracted into the threaded insert 130 that the plunger threads 155 are not engaged with the insert threads 132.

Each of the insert threads 132 include a proximal flank 132a and a distal flank 132b. Each of the plunger threads 155 include a proximal flank 155a and a distal flank 155b. The proximal flanks 132a, 155a are configured to engage when the plunger 150 is threaded into the threaded insert 130. In the illustrated embodiment, the proximal flank 155a of the plunger threads 155 and the proximal flank 132a of the insert threads 132 include an engagement angle β that may range from about 45 degrees to about 90 degrees and from about 70 degrees to about 80 degrees. A pitch of the threads 132, 155 can range from about 8 threads per inch to about 16 threads per inch. A height of the threads 132, 155 may range from about 0.040 inches to about 0.100 inches. With the plunger threads 155 engaged with the insert threads 132, as shown in FIG. 10A, pressure builds in the fluid reservoir 111 as the thread rail 154 is rotated and a proximally directed force or load acting on the flat head 166 pushes the proximal flank 155a into the proximal flank 132a. This load may then translate to the threaded insert 130 and then to the fluid reservoir 111 through the load transfer member 133 and the load transfer orifice 114.

FIGS. 11A and 11B illustrate an embodiment of the actuator 170. As illustrated, the actuator 170 can include the handle 171 and a plunger guide member 172. The handle 171 and plunger guide member 172 can be formed of a rigid or semi-rigid material, such as polycarbonate, polyethylene, polypropylene, nylon, acrylonitrile butadiene styrene, nylon, polysulfone, polyetheretherketone or polyoxymethylene. In some embodiments, the materials may be reinforced or filled with a filler, such as glass. Other materials are contemplated. In certain embodiments, the handle 171 and the plunger guide member 172 include a unibody construct. In other embodiments, the handle and the plunger guide member 172 include separate components that are configured to be assembled together. In some embodiments, the handle 171 may include an over molded, compliant material to improve handling of the handle 171. The handle 171 can include a bore 184. Channels 185 may be disposed within the bore 184 to engage with the ribs 153 of the trigger grip 151 to maintain axial alignment of the handle 171 with the trigger grip 151 as the handle 171 is displaced over the trigger grip 151. Further, a proximal actuator ramp 173 can be disposed within the bore 184 to engage with the proximal plunger ramp 152 of the plunger 150 when the handle 171 is displaced distally relative to the trigger grip 151.

The plunger guide member 172 can extend distally from the handle 171. The plunger guide member 172 can include a first rail 178, a second rail 179, and a middle rail 176 that define an E-shape channel 186 including a first channel 187 and a second channel 188 separated by the middle rail 176. In another embodiment, the plunger guide member 172 may include the first rail 178 and the second rail 179 that define a U-shape channel. The first channel 187 can receive the first rail 160 and the second channel 188 can receive the second rail 161 of the thread rail 154. The middle rail 176 can be received within the channel 162 of the thread rail 154. A distal actuator ramp 174 can be disposed at a distal end of each of the rails 176, 178, 179 to engage with the distal plunger ramp 157 when the plunger guide member 172 is distally displaced relative to the thread rail 154. In other embodiments, one or more intermediate actuator ramps can be disposed along the length of the plunger guide member 172 proximal to the distal actuator ramp 174. The intermediate actuator ramps can be configured to engage with one or more intermediate plunger ramps disposed along the length of the plunger 150. The intermediate actuator ramps may facilitate displacement of an intermediate portion of the plunger guide member 172 radially inward. A resilient member 177 (e.g., compression spring) may be disposed around a proximal portion of the plunger guide member 172. The resilient member 177 can be a compression coil spring to provide a return force to the handle 171. Other types of resilient members are contemplated within the scope of this disclosure.

The first channel 187 may include support ramps 189 disposed along a length of the channel 187 with slots or gaps 181 disposed between adjacent support ramps 189. The second channel 188 may include support ramps 182 disposed along a length of the channel 188 with slots or gaps 183 disposed between adjacent support ramps 182. The support ramps 189 and the support ramps 182 may be axially offset. In other words, as shown in FIG. 11B, the support ramp 189 is transversely aligned with the slot 183. The support ramps 182, 189 can engage with protrusions 156, 164 of the thread rail 154 to maintain engagement of the plunger threads 155 with the insert threads 132 when the inflation device 100 is in a pressurization state. The slots 181, 183 can receive the protrusions 156, 164 to allow the plunger threads 155 to disengage with the insert threads 132 when the inflation device 100 is in a depressurization state.

In use, the inflation device 100 may be utilized to inflate an inflatable medical device (e.g., balloon). FIG. 12A illustrates the inflation device 100 in the pressurization state. As illustrated, a proximal end of a tubing 102 may be coupled to the nozzle 113 of the pressure member 110 and a distal end of the tubing 102 can be coupled to the balloon (not shown). The tubing 102 may be in fluid communication with the fluid reservoir 111 and the balloon. A fluid (e.g., saline) can be disposed within the fluid reservoir 111. The plunger tip 120 can be disposed within the fluid reservoir 111 and operatively coupled to the thread rail 154 of the plunger 150. The thread rail 154 may be slidingly disposed within the plunger guide member 172 of the actuator 170 such that the protrusions 156 of the thread rail 154 are engaged with the support ramps 189 of the plunger guide member 172 and protrusions 164 (not shown) of the thread rail 154 are engaged with support ramps 182 (not shown) of the plunger guide member 172 to maintain engagement of the plunger threads 155 with the insert threads 132 of the threaded insert 130.

While in the pressurization state, the handle 171 may be rotated by a first hand of a user in a clockwise direction to displace the plunger tip 120 distally while grip 140 is grasped by a second hand of the user to prevent the fluid reservoir 111 from rotating. When the handle 171 is rotated, the thread rail 154 and the plunger guide member 172 are rotated relative to the threaded insert 130 causing the plunger tip 120 to be distally displaced by way of a threaded engagement of the plunger threads 155 with the insert threads 132. As the plunger tip 120 is displaced distally, the fluid within the fluid reservoir 111 can be pushed into the tubing 102 and the balloon to inflate the balloon. Continued rotation of the handle 171 can cause pressure within the fluid reservoir 111 to increase as resistance to inflation is exerted by the balloon. The pressure within the fluid reservoir 111 can apply a proximally directed force or load to the plunger threads 155 and the insert threads 132. The proximally directed force can be transferred from the insert threads 132 to the fluid reservoir 111 through the load transfer members 133 and load transfer orifices 114. The pressure within the fluid reservoir 111 can be increased up to about 100 atmospheres. In certain embodiments, the reservoir pressure can be measured by a pressure gage, such as the pressure gage 112.

FIG. 12B illustrates the inflation device 100 in the depressurization state. In the depressurization state, the plunger threads 155 can be disengaged from the insert threads 132 allowing the plunger 150 to freely axially translate relative to the fluid reservoir 111. In the illustrated embodiment, the actuator 170 can be displaced distally relative to the plunger 150 when a user applies a distally directed force to the handle 171. In some embodiments, the distally directed force may be applied by a hand of the user. In another embodiment, the distally directed force can be applied when the handle 171 is forced against a rigid surface, such as a tabletop. The handle 171 may be distally displaced over the trigger grip 151 causing the plunger guide member 172 to be distally displaced relative to the thread rail 154. The support ramps 189 may be displaced distally relative to the protrusions 156, wherein the protrusions 156 are received in the slots 181 to allow the thread rail 154 to be displaced downward perpendicular to the longitudinal axis of the inflation device 100.

The proximal actuator ramp 173 (not shown) can engage the proximal plunger ramp 152 (not shown) and the distal actuator ramp 174 can engage the distal plunger ramp 157 to displace the thread rail 154 radially inward such that the plunger threads 155 disengage from the insert threads 132. The downward displacement of the thread rail 154 causes the proximally directed force applied to the threaded insert 130 and the fluid reservoir 111 to be released. A release force applied to the actuator 170 can range from about 2 pounds of force to about 12 pounds of force. Further, the distal end 159 of the thread rail 154 can be displaced downward within the plunger tip 120 relative to the coupling pin 123. When the plunger threads 155 are disengaged from the insert threads 132, the plunger 150 and the actuator 170 can be freely moved axially resulting in depressurization of the fluid reservoir 111 and the balloon.

In certain embodiments, the free axial movement of the plunger 150 may allow the tubing 102 to be quickly primed and allow air bubbles to be removed from the fluid reservoir 111. Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of pressurizing and depressurizing an inflation device may include one or more of the following steps: radial outwardly displacing a thread rail to engage a threaded insert, wherein protrusions of a thread rail engage with support ramps of a guide member; distally displacing a plunger toward a distal end of a fluid reservoir to pressurize a fluid within the fluid reservoir; actuating an actuator to distally axially displace a guide member relative to the thread rail; radial inwardly displacing the thread rail to disengage the thread rail from the threaded insert, wherein the protrusions disengage from the support ramps and the guide member engages with a plunger ramp; and proximally displacing the plunger to depressurize the fluid within the fluid reservoir. Other steps are also contemplated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The phrase “coupled to” refers to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest to the practitioner during use.

“Fluid” is used in its broadest sense, to refer to any fluid, including both liquids and gases as well as solutions, compounds, suspensions, etc., that generally behaves as a fluid.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments the feature can have a precisely perpendicular configuration.

The terms “a” and “an” can be described as one, but not limited to one. For example, although the disclosure may recite a plunger tip having “an O-ring,” the disclosure also contemplates that the plunger tip can have two or more O-rings.

Unless otherwise stated, all ranges include both endpoints and all numbers between the endpoints.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the invention.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. An inflation device, comprising:
a pressure member comprising:
a fluid reservoir, wherein the fluid reservoir comprises a load transfer orifice;
a threaded insert comprising:
a load transfer member configured to be disposed within the load transfer orifice; and
internal threads;
a plunger comprising:
a trigger grip;
a thread rail extending distally from the trigger grip and comprising:
external threads configured to engage with the internal threads of the threaded insert; and
protrusions extending downward from the thread rail; and
a plunger tip coupled to a distal end of the thread rail; and
an actuator comprising;
a handle operably coupled to the trigger grip; and
a guide member extending distally from the handle and comprising:
support ramps configured to engage with the protrusions of the thread rail; and
slots disposed between the ramps configured to receive the protrusions.

2. The inflation device of claim 1, wherein the load transfer member and the load transfer orifice are configured to transfer an axial load from the internal threads of the threaded insert to the fluid reservoir.

3. The inflation device of claim 1, wherein the pressure member further comprises a pressure gage in fluid communication with the fluid reservoir to measure a fluid pressure within the fluid reservoir.

4. The inflation device of claim 1, wherein the fluid reservoir further comprises a nozzle to permit transmission of a fluid pressure from the fluid reservoir to an inflatable medical device.

5. The inflation device of claim 1, wherein the plunger tip comprises:
a body comprising:
a circumferential groove;
a pin hole;
an interface member to couple with a distal end of the thread rail; and
a distal end face;
an O-ring disposed within the circumferential groove; and
a pin disposed through the pin hole to couple the thread rail to the plunger tip.

6. The inflation device of claim 5, wherein the distal end face is concave.

7. The inflation device of claim 5, wherein a transition angle from the distal end face to an inter for, surface of the fluid reservoir is greater than 90 degrees.

8. The inflation device of claim 1, wherein the internal threads of the threaded insert and the external threads of the thread rail comprise an engagement angle ranging from degrees to 90 degrees.

9. The inflation device of claim 1, wherein the plunger further comprises:
a distal plunger ramp disposed adjacent a distal end of the thread rail;
a proximal plunger ramp disposed adjacent the trigger grip; and
a U-shape channel of the thread rail to receive the guide member.

10. The inflation device of claim 1,
wherein the protrusions are disposed in a first column and a second column along a length of the thread rail;
wherein each of the protrusions in the first column is longitudinally offset from an adjacent protrusion in the first column, wherein a gap is disposed between adjacent protrusions of the first column;
wherein each of the protrusions in the second column is longitudinally offset from an adjacent protrusion in the second column, wherein a gap is disposed between adjacent protrusions of the second column; and
wherein each of the protrusions in the first column is longitudinally offset from an adjacent protrusion in the second column.

11. The inflation device of claim 1, wherein the actuator further comprises:
a channel of the guide member to slidingly receive the thread rail;
a distal actuator ramp disposed adjacent a distal end of the guide member to engage with the distal plunger ramp;
a proximal actuator ramp disposed adjacent the handle to engage with the proximal plunger ramp; and
support ramps disposed within the guide member.

12. The inflation device of claim 11,
wherein the support ramps are disposed in a first column and a second column along a length of the guide member,
wherein each of the support ramps in the first column are longitudinally offset from an adjacent ramp in the first column, wherein a slot is disposed between adjacent support ramps in the first column,
wherein each of the support ramps in the second column are longitudinally offset from an adjacent ramp in the second column, wherein the slot is disposed between adjacent support ramps in the second column, and
wherein each of the support ramps in the first column are longitudinally offset from an adjacent support ramp in the second column.

13. An inflation device, comprising:
a threaded insert comprising a load transfer member;
a plunger comprising an elongate thread rail to selectively engage with the threaded insert, and
an actuator comprising a guide member operatively coupled to the thread rail.

14. The inflation device of claim 13,
wherein the thread rail comprises a plurality of protrusions extending from a bottom surface of the thread rail;
wherein the plurality of protrusions are disposed in a first column and a second column along a length of the thread rail;
wherein each of the plurality of protrusions in the first column is longitudinally offset from an adjacent protrusion in the first column, wherein a gap is disposed between adjacent protrusions;
wherein each of the plurality of protrusions in the second column is longitudinally offset from an adjacent protrusion in the second column, wherein a gap is disposed between adjacent protrusions; and
wherein each of the plurality of protrusions in the first column is longitudinally offset from an adjacent protrusion in the second column.

15. The inflation device of claim 14,
wherein the guide member comprises a plurality of support ramps disposed within a guide channel;
wherein the plurality of support ramps are disposed in a first column and a second column along a length of the guide member;
wherein each of the plurality of support ramps in the first column is longitudinally offset from an adjacent support ramp in the first column, wherein a slot is disposed between adjacent support ramps;
wherein each of the plurality of support ramps in the second column is longitudinally offset from an adjacent support ramp in the first column, wherein the slot is disposed between adjacent support ramps; and
wherein each of the plurality of support ramps in the first column is longitudinally offset from an adjacent support ramp in the second column.

16. The inflation device of claim 15,
wherein the plurality of protrusions engage the plurality of support ramps to engage the thread rail with the threaded insert when the inflation device is in a pressurization state.

17. The inflation device of any one of claim 13, wherein the load transfer member transfers an axial load from the threaded insert to a fluid reservoir when the inflation device is in the pressurization state.

18. The inflation device of claim 15, wherein the plurality of protrusions are disposed within the slots to disengage the thread rail from the threaded insert when the inflation device is in a depressurization state.

19. A method of pressurizing and depressurizing an inflation device, comprising:
radial outwardly displacing a thread rail to engage a threaded insert, wherein protrusions of a thread rail engage with support ramps of a guide member;
distally displacing a plunger toward a distal end of a fluid reservoir to pressurize a fluid within the fluid reservoir;
actuating an actuator to distally axially displace a guide member relative to the thread rail;
radial inwardly displacing the thread rail to disengage the thread rail from the threaded insert, wherein the protrusions disengage from the support ramps and the guide member engages with a plunger ramp; and
proximally displacing the plunger to depressurize the fluid within the fluid reservoir.

20. The method of claim 19, further comprising transferring a proximally directed load applied to the threaded insert to the fluid reservoir, wherein the proximally directed load is transferred through a load transfer member of a threaded insert and a load transfer orifice of the fluid reservoir.

21. The method of claim 19, further comprising coupling a threaded insert to the fluid reservoir, wherein a load transfer member of the threaded insert snap fits with a load transfer orifice of the fluid reservoir.

22. The method of claim 19, further comprising coupling the thread rail to a plunger tip, wherein the thread rail is displaceable perpendicularly relative to a longitudinal axis of the plunger tip.
